(19) United States
(12) Patent Application Publication
(10) Pub. No.: US 2009/0226895 A1
(43) Pub. Date: Sep. 10, 2009

(54) Method of detecting vibrio parahaemolyticus via real-time PCR-hybridization

(75) Inventors: Hang Mei Polly Leung, Hong Kong (HK); Shea Ping Yip, Hong Kong (HK); Shing Shun Tony To, Hong Kong (HK); Lek Hang Constance Lo, Hong Kong (HK)

Correspondence Address: BLANCHARD & ASSOCIATES, 566 WEST ADAMS STREET, SUITE 600, CHICAGO, IL, 60661, US

(73) Assignee: The Hong Kong Polytechnic University, Hong Kong (HK)

(21) Appl. No.: 11/896,499

(22) Filed: Sep. 4, 2007

Related U.S. Application Data
(60) Provisional application No. 60/929,184, filed on Jun. 15, 2007.

(51) Int. Cl. C12Q 1/68

(52) U.S. Cl. 435/6

(57) ABSTRACT

The present invention relates to an assay and methods for determining the presence of pathogenic V. parahaemolyticus in a sample. Determination should be made by detector. Via the assay, rapid, real-time detection of V. parahaemolyticus is possible.

### BACKGROUND

Vibrio parahaemolyticus (V. parahaemolyticus) is a major cause of food-borne gastroenteritis associated with inadequate cooking and consumption of contaminated seafood in South America, Japan, Southeast Asia, India, and Europe. In Hong Kong, V. parahaemolyticus was the most important bacterial pathogen accounting for 32-50% of food-borne infections each year from 2003 to 2006.

Conventional laboratory approach to the detection of V. parahaemolyticus is based on culture and biochemical identification, which are able to detect viable bacteria in the sample. However, these methods usually require three to four days before the results can be finalized. Owing to the laborious and time consuming nature of culture-based detection approach, nucleic acid-based methods have been applied for rapid detection of V. parahaemolyticus. Among various molecular assays, real-time PCR is widely used because of its speed and quantitative nature. Most of the recently published real-time PCR assays employed TaqMan probes for specific detection of V. parahaemolyticus, and detection limits of these real-time assays ranged from 10²-10⁴ CFU/ml of pure culture.

Despite the low detection limit, performance of the real-time assay is often compromised by the low number of target organisms and the presence of inhibitors in the specimens. Incubation of the specimen in enrichment medium from eight hours to overnight is often required before subjecting to real-time assay. After enrichment, the detection limit can be brought to 1 CFU/ml or gram of sample. If the sensitivity of real-time PCR assay is increased, then the duration of enrichment can be shortened, which in turn shortens the turn-around time of the detection procedure.

The product encoded by toxR regulates expression of thermostable direct haemolysin (TDH) of V. paraheamolyticus. All V. parahaemolyticus and a number other Vibrio species carry the toxR gene. The sequence similarity of toxR between different Vibrio species is 52-59%. Thus, species-specific regions within the toxR sequence can be employed for detection of V. parahaemolyticus. Dileep at al reported that there was about 30% increase in the detection rate of V. parahaemolyticus from food and environmental samples when toxR-based PCR was compared with conventional isolation method.

It has been reported that clinical V. parahaemolyticus strains producing TDH are associated with pathogenicity to humans. The haemolysin is encoded by the tdh gene. To date, five types of tdh genes have been identified: tdh1, tdh2, tdh4 and tdh5 are chromosome-borne, and tdh3 is plasmid-borne. These five tdh genes shared 96% DNA sequence similarity. TDH production was mainly contributed by tdh2. The other tdh genotypes were shown to have very low level of gene expression and were less responsible for TDH production. The low level of expression could be caused by one or two base changes within the tdh promoter region. Owing to the big difference in expression level of the tdh genes, the choice of an appropriate tdh marker is important for detection of TDH-producing V. parahaemolyticus.

Rapid identification of V. parahaemolyticus infection facilitates effective tracing of the source and thus the immediate enforcement of public health measures, including food recall, to prevent the spread of infection and reduce the burden of disease in hospitals.

It is an object of the present invention to provide a method for identifying V. parahaemolyticus, as well as overcoming the disadvantages and problems in the prior art.

### DESCRIPTION

The present invention proposes a real-time PCR assay for the detection of pathogenic V. parahaemolyticus. The assay employs two pairs of oligonucleotide primers and two pairs of fluorogenic hybridization probes for the detection of the toxR and tdh2 genes.

The assay is specifically designed for use with commercial detection systems, for example a LightCycler 1.5 System®.

These and other features, aspects, and advantages of the apparatus and methods of the present invention will become better understood from the following description, appended claims, and accompanying drawings where:

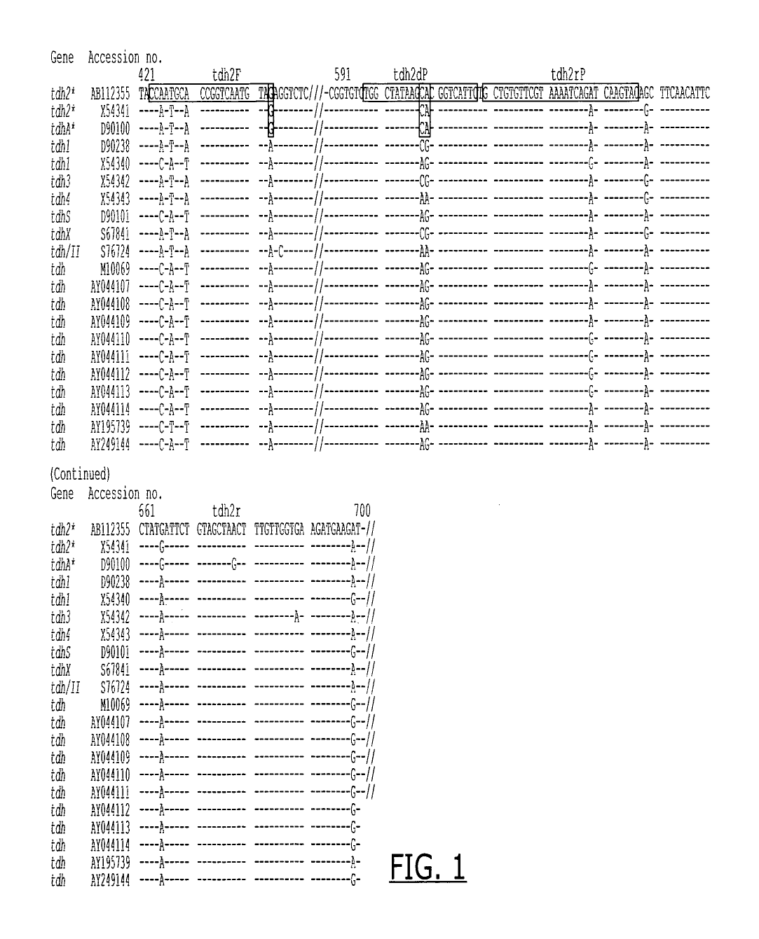

FIG. 1 exhibits tdh2 gene sequence;

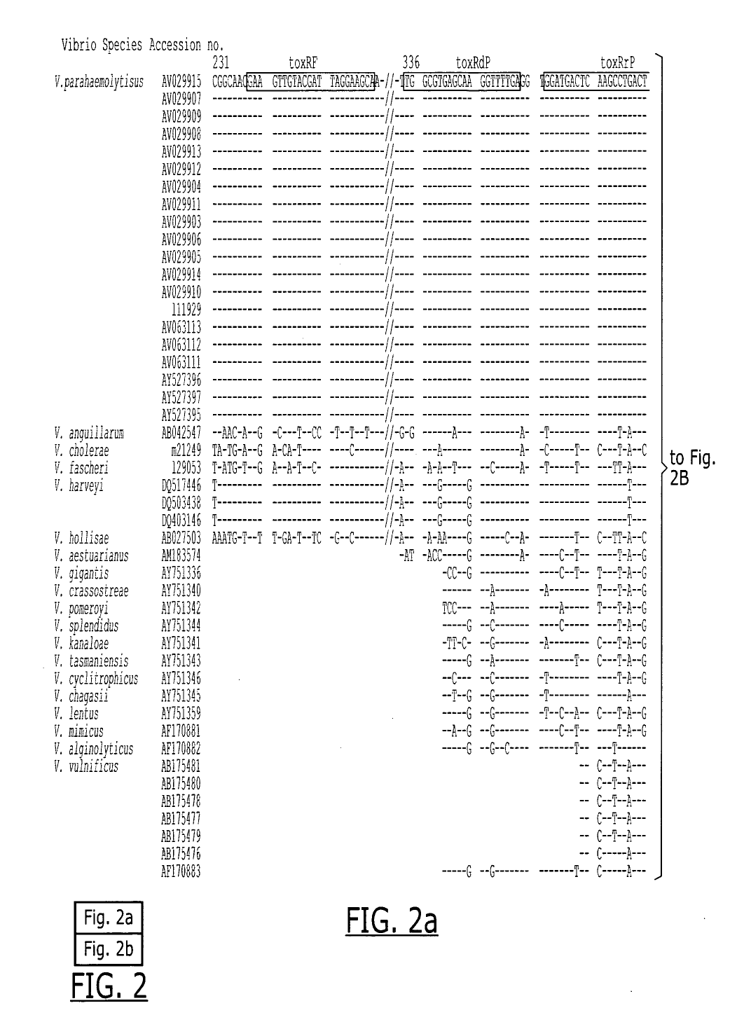

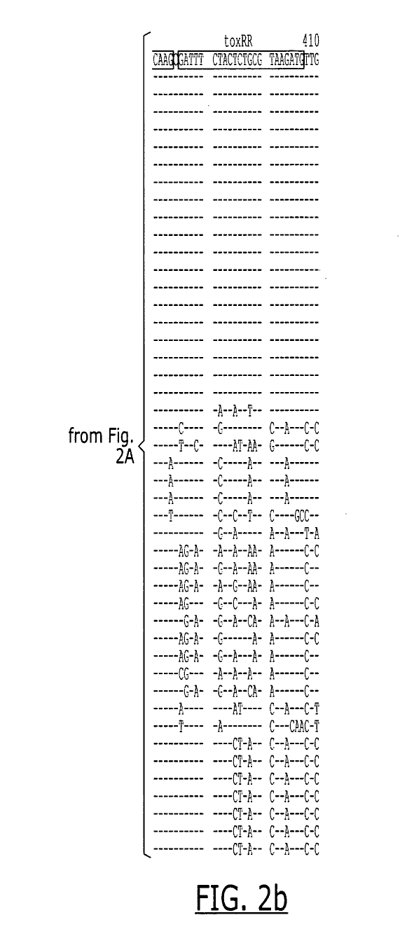

FIG. 2 exhibits toxR gene sequence;

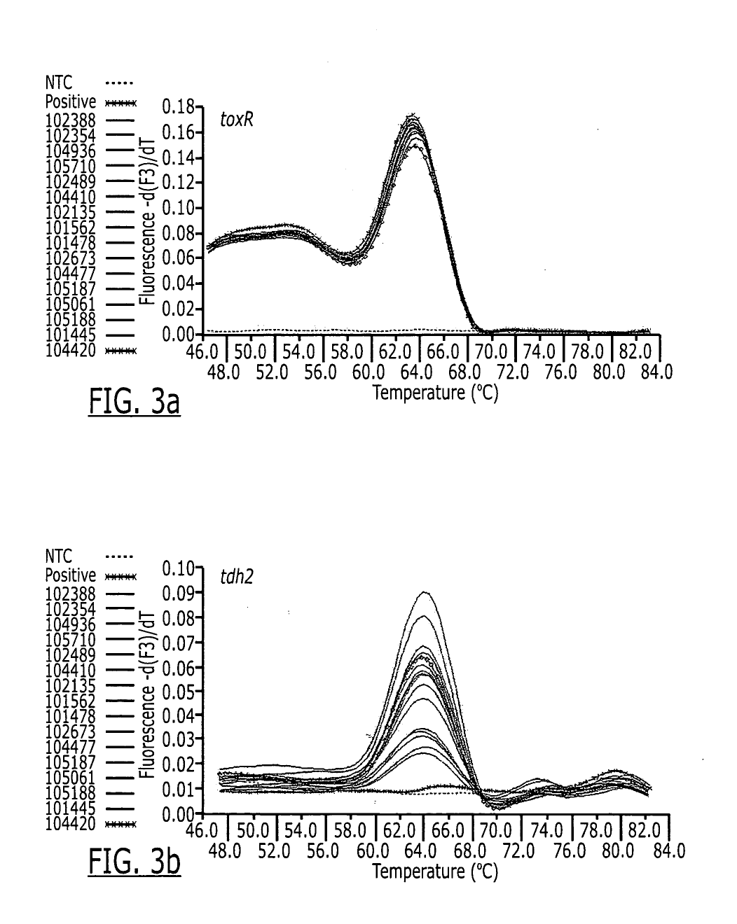

FIG. 3 shows peaks for tdh2 and toxR during melting curve analysis;

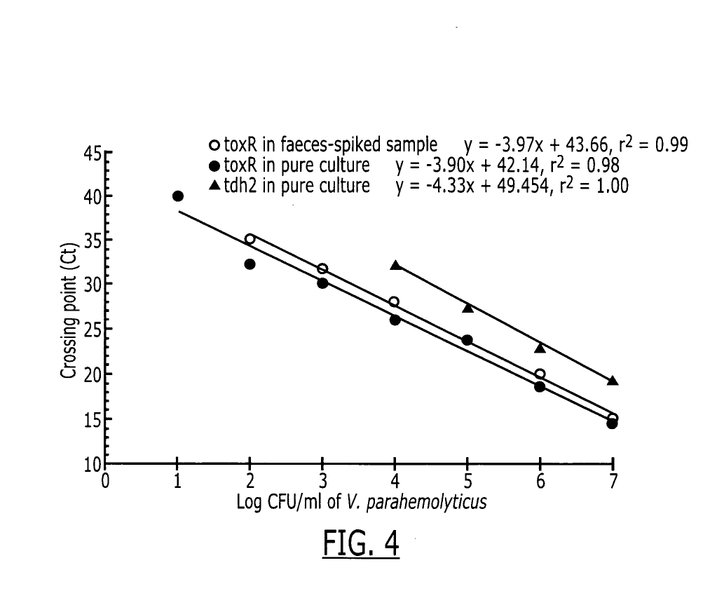

FIG. 4 shows a pure culture of V. parahaemolyticus and stool sample spiked with V. parahaemolyticus subjected to the assay of the present invention.

The following description of certain exemplary embodiment(s) is merely exemplary in nature and is in no way intended to limit the invention, its application, or uses.

Now, to FIGS. 1-4,

The present invention relates to a real-time PCR-hybridization probe assay optimized for rapid and specific detection of V. parahaemolyticus. Primers and hybridization probes targeting a 168-bp V. parahaemolyticus-specific region of toxR gene and a 267-bp region of tdh2 gene were designed.

The present invention also relates to a detection method for V. parahaemolyticus, utilizing the assay of the preset invention and detection devices suitable for rapid detection, for example the LightCycler® system (Roche Diagnostics, Indianapolis, Ind.).

The assay of the present invention includes at least two pairs of oligonucleotide primers and two pairs of fluorogenic hybridization probes for the detection of toxR gene and tdh2 gene, specifically for species V. parahaemolyticus. FIGS. 1 and 2 exhibit tdh2 gene sequence (FIG. 1) and toxR gene sequence (FIG. 2).

The oligonucleotide primers can be selected from the group consisting of SEQ ID NO. 1, SEQ ID NO. 2, SEQ ID NO. 5, and SEQ ID NO. 6. As stated, two primers are included in the assay.

The probes include SEQ ID NO. 3, SEQ ID NO. 4, SEQ ID NO.7, and SEQ ID NO.8. All of the four probes are to be included in the assay. The 3′ end of donor probes are labeled with a fluorophore, such as fluorescein or derivatives thereof. The 5′ ends of tdh2 and toxR recipient probes are preferably labeled with a colorant, for example LC Red 640 and LC Red 705. U.S. Pat. Nos. 4,683,195 and 4,683,202, incorporated herein by reference, discuss methods suitable for preparing the probes of the present invention.

The assay can also contain one or more reagents for detection and/or amplification, such as buffers, divalent cations derived from, for example, magnesium chloride, sodium chloride, potassium chloride, or manganese compounds, serum, for example bovine serum albumin, polymerases including DNA polymerase and RNA polymerase, deoxynucleoside triphosphates, and water such as distilled water, deionized water, or double deionized water. One or more reagents may be used in the assay.

Detection of V. parahaemolyticus by the present invention is accomplished by methods well-known in the art. For example, obtaining a sample, such as bodily fluid, stool, swipe samples, etc., preparation of sample for inclusion into assay, running PCR including but not limited to the steps of initialization, denaturation, annealing, extension/elongation, and determination on whether target DNA is present.

### EXAMPLE

From the bacterial strain, tdh2-positive V. parahaemolyticus UCH-8, 114 strains were isolated from stool specimens of patients suffering from gastroenteritis, 56 strains were isolated from environmental samples, and 16 non-V. parahaemolyticus strains. These strains were cultured on nutrient agar, such cultures then being incubated at 37° C. for 18 to 24 hours.

A fresh bacterial colony was inoculated into 10 ml Luria broth (Oxiod) for non-V. parahaemolyticus strains or alkaline peptone water with 3% salt for V. parahaemolyticus strains. The medium was incubated at 37° C. overnight with agitation at 250 rpm. The overnight broth culture was diluted with normal saline and 10² CFUs were inoculated into 10 ml Luria broth or SAPW. The broth was incubated for 6 hours at 37° C. with agitation at 250 rpm. After incubation, 1 ml of the broth culture was centrifuged at 9000×g for 5 min, the pelleted bacterial cells were washed with 1 ml double-deionized water. The pellet was then resuspended in 100 μL double-deionized water and heated at 100° C. for 5 min. The heated content was centrifuged at 15000×g for 5 min. The supernatant was collected and 1 μL used as DNA template. The DNA templates were either used freshly or stored at −70° C. until use.

Design of primers and probes. Twenty-one tdh and 20 toxR sequences specific for V. parahaemolyticus were retrieved from GenBank database together with 26 toxR sequences from 18 other Vibrio species (see FIGS. 1 and 2). Sequences within each of the tdh and toxR sequence groups were subjected to multiple sequence alignment using Multalign algorithm program. Primers and probes specific for tdh2 and toxR were designed within the conserved regions (SEQ ID Nos. 1-8). Two mismatches were introduced in each of the forward and reverse tdh2 primers. The mismatched bases near the 3′ ends of both primers were introduced to enhance specific hybridization to tdh2 template. The other mismatched bases were introduced in the middle of the primer sequence to minimize the formation of primer dimers. The 3′ ends of donor probes were labeled with fluorescein. The 5′ ends of tdh2 and toxR receipt probes were labeled with LC Red 640 and LC Red 705, respectively. Melting temperature (Tm) of tdh2 and toxR hybridization probes were predicted by TM Utility software version 1.5 (Idaho Technology Inc., Salt Lake City, Utah, USA).

Real-time PCR assay. The real-time PCR mixture consisted of 1×NH₄ buffer (Bioline, Luckenwalde, Germany), 3 mM MgCl₂, 50 μM of each dNTP (PE Applied BioSystems, Foster City, Calif., USA), 1.25 μM of each tdh2 primer, 1.0 μM of each tdh2 probe, 0.32 μM of each toxR probe, 10 μg bovine serum albumin (New England BioLabs, Hertfordshire, UK) and 1 U BIOTAQ DNA polymerase (Bioline, Luckenwalde, Germany), 1 μl of DNA template and double-deionized water to a final volume of 20 μl. Primers were ordered from Qiagen (Hilden, Germany) and probes from Metabion (Martinsried, Deutschland). The PCR reaction mixture was subjected to 50 cycles of amplification using the LightCycler 1.5 System® (Roche Diagnostics). The PCR protocol consisted of DNA denaturation at 94° C. for 5 s, 57° C. for 10 s and 72° C. for 15 s. Signals of fluorescent probes were measured during melting curve analysis, the PCR products were heated to 95° C. without hold, cooled to 40° C. (20° C./s) for 30 s, then heated slowly (0.1° C./s) to 90° C., and finally cooled 40° C. (20° C./s). The melting temperatures specific to the probes were also measured. The actual Tm of a particular hybridization probe was compared with the predicted Tm. The specificity of amplicons were confirmed by agarose gel electrophoresis and sequencing using the ABI PRISM™ 310 Genetic Analyzer (PE Applied BioSystem).

Standard curve for the real-time PCR assay. A fresh colony from the control V. parahaemolyticus strain UCH-8 was inoculated into 10 ml SAPW, and incubated overnight at 37° C. with agitation at 250 rpm. After incubation, 10² CFUs of V. parahaemolyticus were inoculated again into 10 ml SAPW, followed by incubation at 37° C. for 6 hours. Ten-fold serial dilutions (from 10⁷ to 10⁰ CFU/ml) were prepared from the 6-hr culture using normal saline. One milliliter of content was drawn from each dilution and centrifuged at 9000×g and DNA then extracted as described above. One microliter of DNA was used as template for the real-time PCR assay. Threshold cycle (Cₜ) was plotted against log CFU/ml.

Effect of fecal materials on the performance of the real-time PCR assay. Five grams of feces from a healthy donor was suspended in 10 ml double-deionized water. One-milliliter aliquots of fecal suspension were spiked with V. parahaemolyticus UCH-8 to final concentrations of 10⁷ to 10⁰ CFU/ml. Bacterial DNA was extracted directly from each of the fecal suspension as described above.

Effect of enrichment on performance of the real-time PCR assay. In order to assess the enrichment effect on the real-time assay, 10² CFUs of V. parahaemolyticus were added to 1 ml fecal suspension and incubated in 10 ml of SAPW at 37° C. with agitation at 250 rpm. One milliliter of the enriched content was withdrawn for DNA extraction at every 1-hour interval up to 8 hours. Fourteen tdh2-positive V. parahaemolyticus strains were randomly selected for this part of the study.

FIGS. 3(a and b) show distinctive peaks for toxR (a) and tdh2 (b) during melting curve analysis. The fluorescence signal emitted from fluorescein of recipient toxR probe was stronger that that from recipient tdh2 probe. The actual Tm of all V. parahaemolyticus strains measured during the melting curve analysis were 63.7±0.2° C. for tdh2 probe and 63.6±0.2° C. for toxR probe. The measured Tm of both loci were very close to the predicted ones (see Sequence ID Nos. 1-8). Presence of amplicons was confirmed by agarose gel electrophoresis and DNA sequencing.

Significant fluorescent signals from toxR gene were detected from all clinical and environment V. parahaemolyticus stains tested. The tdh2 gene was detected in 90.4% (103/114) of clinical V. parahaemolyticus strains. On the other hand, only 3.6% (2/56) of the environmental strains produced weak signal with tdh2-specific probes. The signal difference between clinical and environmental samples could be due to variation in the amount of DNA loaded. No fluorescence signal was generated from both toxR and tdh2 probes by bacterial species other than V. parahaemolyticus.

FIG. 4 exhibits a pore culture of V. parahaemolyticus subjected to the real time PCR assay: the dynamic range was 10⁷ to 10¹ CFU/ml for toxR gene and 10⁷ to 10⁴ CFU/ml for tdh2 gene.

A good linear relationship was demonstrated between bacterial count and threshold cycle for both target genes (r²=0.98 for toxR, r²=0.99 for tdh2). When toxR is considered, the assay was able to detect one CFU per reaction (1000 CFU/ml) in 30 cycles.

In order to assess the effect of fecal materials on the efficiency of the real-time PCR assay, V. parahaemolyticus was spiked into fecal suspension. The dynamic range of the assay for toxR was 10⁷ to 10² CFU/ml (see FIG. 4), and hence the sensitivity was 10-fold lower than that of pure V. parahaemolyticus culture. The Cₜ values also increased slightly by 0.1 to 2.9 cycles. A single CFU per reaction was detected in 32 cycles. However, no fluorescence signal was detected from tdh2 probe in any of the spiked samples. In order to assess the effect of enrichment on the assay, 10² CFUs of V. parahaemolyticus were seeded into fecal suspension and enriched in SAPW for different duration. Fluorescence signal was not detected during the first 4 hours. After 5-hour enrichment, fluorescence signals from both toxR and tdh2 probes were detected in only six out of 14 (43%) tested clinical V. parahaemolyticus isolates. Besides, non-specific fluorescence signals were detected in all 14 tested clinical V. parahaemolyticus strains, the Cₜ values were 14.0 and 23.0 for toxR and tdh2, respectively.

The present invention relates to a real-time PCR assay and detection method targeting toxR and tdh2, developed for use with a detection system, for example LightCyler 1.5® or LightCycler 2.0®. The present invention is capable of allowing the detection of V. parahaemolyticus from pure culture and fecal samples. The assay of the invention has a wide dynamic range of detection for toxR (10⁷-10¹ CFU/ml) and is able to detect a single CFU per reaction within 30 cycles. In the presence of fecal materials, the detection limit and Cₜ value of toxR is only slighted affected. Specific signal from the tdh2 probe can be detected in as few as 6 hour enrichment.

The presence of amplicons can be detected by the pairs of fluorogenic hybridization probes of the assay. While not to be found by theory, upon excitation of the donor fluorophore, energy will be optimally transferred to the recipient fluorophore when the probes are 1-4 bases apart. Fluorescence will be emitted from the recipient fluorophore of a specific wavelength. This distance-development energy transfer process reduces the background fluorescence and increases the specificity of the assay.

The turn around time when using the present assay is between 75 minutes to 90 minutes, including a reaction time between 45 minutes to 55 minutes and a DNA extraction procedure between 25 minutes to 35 minutes. Together with an enrichment step, the assay requires between 6 hours to 8 hours for the entire detection process.

1. A method of detecting Vibrio parahaemolyticus, comprising the steps of:
obtaining a sample,
preparing sample for inclusion into assay,
running real-time PCR, and
detecting toxR gene and tdh2 gene,
wherein running real-time PCR uses an assay having two pairs of oligonucleotide primers and two pairs of probes.

2. The method of detecting Vibrio parahaemolyticus of claim 1, wherein said probes are labeled on the 3′ end with a fluorophore.

3. The method of detecting Vibrio parahaemolyticus of claim 1, wherein the 5′ end of said toxR gene and said tdh2 gene are labeled with a colorant.

4. The method of detecting Vibrio parahaemolyticus of claim 2, wherein said fluorophore is a fluorescein or derivative thereof.

5. The method of detecting Vibrio parahaemolyticus of claim 2, wherein said probes comprise SEQ ID NO. 3, SEQ ID NO. 4, SEQ ID NO. 7, and SEQ ID NO. 8.

6. The method of detecting Vibrio parahaemolyticus of claim 1, wherein said primers are selected from the group consisting of SEQ ID NO. 1, SEQ ID NO. 2, SEQ ID NO. 5, and SEQ ID NO. 6.

7. The method of detecting Vibrio parahaemolyticus of claim 1, wherein said sample is stool.

8. The method of detecting Vibrio parahaemolyticus of claim 1, wherein running real-time PCR occurs on a detection device.

9. An assay for rapid detection of Vibrio parahaemolyticus comprising two pairs of oligonucleotide primers selected from the group consisting of SEQ ID NO. 1, SEQ ID NO. 2, SEQ ID NO. 5, and SEQ ID NO. 6, and two probes consisting of SEQ ID NO. 3, SEQ ID NO. 4, SEQ ID NO. 7, and SEQ ID NO. 8.

10. The assay for rapid detection of Vibrio parahaemolyticus of claim 9, further comprising one or more reagents selected from buffers cations, serum, polymerases, deoxynucleoside triphosphates, and water.


SEQUENCE LISTING (8 sequences)

SEQ ID NO: 1 - DNA, 21 bp, Unknown
  Description of Unknown Tdh2 sequence
ccaatgcacccgtcaatctag

SEQ ID NO: 2 - DNA, 25 bp, Unknown
  Description of Unknown Tdh2 sequence
caccaacaaagttacctacagtatc

SEQ ID NO: 3 - DNA, 21 bp, Unknown
  Description of Unknown Tdh2 sequence
tggctataagcacggtcattc

SEQ ID NO: 4 - DNA, 28 bp, Unknown
  Description of Unknown Tdh2 sequence
gctgtgttcgtaaaatcagatcaagtac

SEQ ID NO: 5 - DNA, 22 bp, Unknown
  Description of Unknown ToxR sequence
gaagttgtacgattaggaagca

SEQ ID NO: 6 - DNA, 22 bp, Unknown
  Description of Unknown ToxR sequence
catcttacgcagagtagaaatc

SEQ ID NO: 7 - DNA, 20 bp, Unknown
  Description of Unknown ToxR sequence
tggcgtgagcaaggttttga

SEQ ID NO: 8 - DNA, 23 bp, Unknown
  Description of Unknown ToxR sequence
ggatgactcaagcctgactcaag